SEQUENCE LISTING (29 sequences)

SEQ ID NO: 1 - DNA, 25 bp, Artificial Sequence
  Primer
tcacccacactgtgcccatctacga

SEQ ID NO: 2 - DNA, 25 bp, Artificial Sequence
  Primer
cagcggaaccgctcattgccaatgg

SEQ ID NO: 3 - DNA, 25 bp, Artificial Sequence
  Probe
atgccccccccatgccatcctgcgt

SEQ ID NO: 4 - DNA, 20 bp, Artificial Sequence
  Primer
gccaaccatgctcaacttcg

SEQ ID NO: 5 - DNA, 24 bp, Artificial Sequence
  Primer
tgcagttttgttccaggaatacat

SEQ ID NO: 6 - DNA, 24 bp, Artificial Sequence
  Probe
tgcctctctccagcagactgcgga

SEQ ID NO: 7 - DNA, 16 bp, Artificial Sequence
  Primer
tgcccccaaactcccc

SEQ ID NO: 8 - DNA, 21 bp, Artificial Sequence
  Primer
acatagctgggcatcagctgt

SEQ ID NO: 9 - DNA, 25 bp, Artificial Sequence
  Probe
aagctcaggcaagtgtgattcgtca

SEQ ID NO: 10 - DNA, 21 bp, Artificial Sequence
  Primer
ccaaagctcaggcaacaagtg

SEQ ID NO: 11 - DNA, 20 bp, Artificial Sequence
  Primer
ttgggcaggtctggtggtta

SEQ ID NO: 12 - DNA, 29 bp, Artificial Sequence
  Probe
attcgtcatacagctgatgcccagctatg

SEQ ID NO: 13 - DNA, 28 bp, Artificial Sequence
  Primer
caagacatactttaaaagttcccatctg

SEQ ID NO: 14 - DNA, 24 bp, Artificial Sequence
  Primer
ctttcacaacctttccagctacag

SEQ ID NO: 15 - DNA, 21 bp, Artificial Sequence
  Probe
aggcccacacgaggacgcaca

SEQ ID NO: 16 - DNA, 23 bp, Artificial Sequence
  Primer
gaatcttacgggaagatcaaccc

SEQ ID NO: 17 - DNA, 19 bp, Artificial Sequence
  Primer
cgcagagtcggctaaggtg

SEQ ID NO: 18 - DNA, 21 bp, Artificial Sequence
  Probe
agctggtgtgctgcaacccca

SEQ ID NO: 19 - DNA, 24 bp, Artificial Sequence
  Primer
agagagttgagacaccagttttgc

SEQ ID NO: 20 - DNA, 23 bp, Artificial Sequence
  Primer
atagtcatccagaggcggaagtt

SEQ ID NO: 21 - DNA, 24 bp, Artificial Sequence
  Probe
agtgccccgacacaccgagatcct

SEQ ID NO: 22 - DNA, 26 bp, Artificial Sequence
  Primer
gcctgtcgattatacagatgatgaaa

SEQ ID NO: 23 - DNA, 21 bp, Artificial Sequence
  Primer
cgctgcccagtgttcattact

SEQ ID NO: 24 - DNA, 33 bp, Artificial Sequence
  Probe
agaagaatgaatcatcctcagctagccactgct

SEQ ID NO: 25 - protein, 480 aa, Homo Sapiens
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 26 - protein, 20 aa, Homo Sapiens
SERPKESMYSWNKTAEKSDF

SEQ ID NO: 27 - protein, 13 aa, Homo Sapiens
PPYSPSDFEPSQV

SEQ ID NO: 28 - protein, 20 aa, Homo Sapiens
FKEEEKPVSAPKLPKAQATS

SEQ ID NO: 29 - protein, 18 aa, Homo Sapiens
SAAKVTPQIDSSRIRSHI

(19) United States
(12) Patent Application Publication
(10) Pub. No.: US 2003/0198961 A1
(43) Pub. Date: Oct. 23, 2003

(54) Determining cancer aggressiveness

(76) Inventors: Thomas C. Spelsberg, Rochester, MN (US); Patrick C. Roche, Rochester, MN (US); Monica Reinholz, Rochester, MN (US); Steven A. Johnsen, Rochester, MN (US); Malayannan Subramaniam, Zumbrota, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/123,626

(22) Filed: Apr. 15, 2002

(51) Int. Cl. C12Q 1/68; G01N 33/574; C07H 21/04; C12P 19/34; C07K 16/40

(52) U.S. Cl. 435/6; 435/7.23; 435/91.2; 530/388.26; 536/24.3

(57) ABSTRACT

The invention provides materials and methods for determining the aggressiveness of a cancer in a mammal. Specifically, the invention provides methods and methods for measuring the level of a TIEG marker in a sample. Such levels can be correlated with the aggressiveness of a cancer to predict patient outcome and develop treatment regimens.

### STATEMENT AS TO FEDERALLY SPONSORED RESEARCH

Funding for the work described herein was provided by the federal government (National Institutes of Health Grant No.'s AR 43627, DE 14036 and CA 15083), which may have certain rights in the invention.

### BACKGROUND

1. Technical Field

The invention relates to methods and materials involved in determining cancer aggressiveness.

2. Background Information

Optimum management of breast cancer patients requires a multidisciplinary approach that includes the use of certain tumor markers. Currently, it is difficult to reliably identify high-risk patients (i.e., those patients that will need adjuvant chemotherapy) and low-risk patients (i.e., those patients that can be spared adjuvant chemotherapy) by traditional histomorphologic and clinical characteristics, such as tumor size, histologic grade, age, steroid hormone receptor status or menopausal status. These characteristics are used to select therapies for patients and up to 90% of node-negative breast cancer patients would be candidates for adjuvant chemotherapy. While most patients with node-negative breast cancer are cured by locoregional treatment, about 30% will relapse and need adjuvant chemotherapy. Because traditional histomorphometric and clinical factors fail to identify the high-risk patients who may benefit from adjuvant chemotherapy, other prognostic factors are needed.

### SUMMARY

It has been discovered that measuring levels of TIEG nucleic acids or polypeptides in patient samples can provide useful information about the aggressiveness of a cancer in that patient. It also has been discovered that measuring levels of TIEG nucleic acids or polypeptides in combination with levels of nucleic acids or polypeptides of biomolecules regulated by TIEG can provide additional useful information about cancer aggressiveness.

In general, the invention features a method for determining the aggressiveness of a cancer in a mammal. The method includes determining, in a test sample from the mammal, the presence or absence of a TIEG marker and correlating that presence or absence with aggressiveness of the cancer, for example a breast cancer. The test sample can be, for example, a tumor biopsy from a mammal such as a human. The test samples typically are chosen to reflect an adequate sampling and presence of cancer cells. For carcinomas (e.g., breast cancers), for example, it is desirable that the sample contain at least 60% epithelial cells. The presence of the marker can indicate that the cancer is aggressive, or metastatic, whereas the absence of the marker can indicate that the cancer is not aggressive. The marker can be, for example, a reduced level of TIEG RNA, a reduced level of Smad 2 RNA, an elevated level of Smad 7 RNA, or a reduced level of BARD-1 RNA, all in comparison to an appropriate control level of the corresponding RNA. Where appropriate, the marker can be adjusted to reflect the epithelial cell content of the test sample.

Determining the presence or absence of various markers in combination, e.g., combinations of two, three, or more TIEG markers, also can provide useful information on aggressiveness. Again, measurements are typically made with respect to an appropriate control level. For example, two-marker combinations can include: a reduced level of TIEG RNA and a reduced level of Smad 2; a reduced level of TIEG RNA and an elevated level of Smad 7 RNA; a reduced level of TIEG RNA and a reduced level of BARD-1 RNA; a reduced level of BARD-1 and an elevated level of Smad 7; and a reduced level of Smad 2 RNA and an elevated level of Smad 7. Useful three-marker combinations include: a reduced level of TIEG, a reduced level of BARD-1 and a reduced level of SMAD-2 RNA; a reduced level of TIEG RNA, a reduced level of BARD-1 RNA and an elevated level of SMAD-7 RNA; and a reduced level of TIEG RNA, a reduced level of SMAD-2 RNA, and an elevated level of SMAD-7.

The markers can be measured in comparison to baselines established for particular types of cancers. For example, when the marker is a reduced level of TIEG RNA and the cancer is breast cancer, the baseline can be from about 89 to about 100 mRNA molecules ×1000 per picogram of beta-actin (β-actin) mRNA.

In another aspect, the invention features an article of manufacture including an oligonucleotide primer pair that specifically amplifies all or a portion of a target region of a TIEG nucleic acid, where the target region is defined by nucleotides 1-500 (e.g., nucleotides 80-188) of the 5′ portion of the TIEG nucleic acid. The article of manufacture further can include a label or package insert indicating that a level of a TIEG nucleic acid in a test sample from a mammal can be correlated with aggressiveness of a cancer in that mammal.

In another embodiment, the invention features an article of manufacture including a first and a second oligonucleotide primer pair, where the first primer pair amplifies a first target nucleic acid and the second primer pair amplifies a second target nucleic acid in an amplification reaction, with the first and second target nucleic acids being TIEG marker-related nucleic acids. For example, the first target nucleic acid can be a TIEG nucleic acid, and the second target nucleic acid can be a Smad 2, Smad 7 or BARD-1 nucleic acid. The second nucleic acid can be any nucleic acid encoding a biomolecule regulated by TIEG. The article of manufacture further can include a third oligonucleotide primer pair, which amplifies another TIEG marker-related nucleic acid. In such three-combination embodiments, the first target nucleic acid can be a TIEG nucleic acid, the second a BARD-1 or Smad 2 nucleic acid, and the third a Smad 7 nucleic acid. The article of manufacture further can include an oligonucleotide probe specifically hybridizable to a TIEG marker-related nucleic acid. In addition, the article of manufacture can include a label or package insert indicating that a level of a TIEG marker-related nucleic acid in a test sample from a mammal can be correlated with aggressiveness of a cancer in that mammal. Such labels also can include a baseline level or levels of a TIEG marker established for particular types of cancer, and can further indicate that a test sample (e.g., tumor biopsy) can be adjusted for epithelial cell content prior to determining the level of a TIEG marker-related nucleic acid in the test sample.

In another aspect, the invention features an antibody having specific binding affinity for a TIEG polypeptide. The amino acid sequence of the TIEG polypeptide can include a sequence of one of SEQ ID NO's 25, 26, 27, 28, or 29.

In another aspect, the invention features a method for determining the aggressiveness of a cancer in a mammal, including contacting an antibody described above with a test sample from the mammal, detecting the presence or absence of complexes between the antibody and any TIEG polypeptide present in the sample, and correlating the presence or absence of such complexes with aggressiveness of the cancer. This can be combined, if desired, with determining the presence or absence of a TIEG marker other than TIEG polypeptide in the sample.

In another aspect, the invention features a method of assisting a person in determining the aggressiveness of a cancer in a mammal, the method including:

a) determining the presence or absence of a TIEG marker in a sample from the mammal; and,

b) communicating information about the presence or absence of the marker in the sample to that person.

In another aspect, the invention features a method for determining the prognosis of a mammal having a cancer, the method including determining, in a test sample from the mammal, the presence or absence of a TIEG marker and correlating such presence or absence with the prognosis. For example the presence of a TIEG marker can indicate that the prognosis is a bad outcome, whereas the absence of a TIEG marker can indicate that prognosis is a good outcome.

In another aspect, the invention features a method for diagnosing cancer in a mammal, the method including determining, in a test sample from the mammal, the presence or absence of a TIEG marker and correlating such presence or absence with the diagnosis. For example the presence of a TIEG marker (e.g., a reduced level of TIEG RNA compared to a control level of TIEG RNA) can indicate a diagnosis of cancer, whereas the absence of a TIEG marker can indicate a diagnosis of no cancer.

In another aspect, the invention features a method for determining the aggressiveness of a cancer in a mammal, including determining, in a test sample from the mammal, the level of a TIEG biomolecule together with the level of a biomolecule regulated by TIEG, and correlating those levels with aggressiveness.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a bar graph depicting the average TIEG gene expression in normal, non-invasive (DCIS), invasive, and metastatic breast tissue. TIEG gene expression is shown as a percentage of TIEG gene expression in normal breast tissue. Standard errors of the mean (SEM) are presented as error bars. *: p≦0.06 by Student's t-test, p<0.09 by Wilcoxon Rank-Sum test versus normal; **: p≦0.006 by Student's t-test, p<0.009 by Wilcoxon Rank-Sum test versus normal; ***: p<0.0005 by Student's t-test, p<0.0003 by Wilcoxon-Rank Sum test versus normal. Sample size for each group is shown in parentheses.

FIG. 2 is a bar graph depicting the average TIEG gene copy number in normal, non-invasive (DCIS), invasive, and metastatic breast tissue. TIEG gene copy number (number of mRNA molecules) is shown as TIEG gene copy number in normal breast tissue×1000/pg β-actin mRNA. SEMs are presented as error bars. *: p≦0.06 by Student's t-test, p<0.09 by Wilcoxon Rank-Sum test versus normal; **: p≦0.006 by Student's t-test, p<0.009 by Wilcoxon Rank-Sum test versus normal; ***: p<0.0005 by Student's t-test, p<0.0003 by Wilcoxon-Rank Sum test versus normal. Sample size for each group is shown in parentheses.

FIG. 3A is a bar graph depicting TIEG gene expression in N− and N+ primary tumor breast tissue. Average TIEG gene expression is shown as a percentage of TIEG expression in normal breast tissue. SEMs are presented as the error bars. *: p≦0.04 by Student's t-test, p<0.02 by Wilcoxon Rank-Sum test versus normal; **: p≦0.00001 by Student's t-test, p<0.00004 by Wilcoxon Rank-Sum test versus normal. Sample size for each group is shown in parentheses. FIG. 3B is a bar graph depicting individual tumor TIEG gene copy number in N− and N+ primary breast cancers. TIEG gene copy number is shown as TIEG gene copy number in normal breast tissue number×1000/pg β-actin mRNA.

FIG. 4A is a bar graph depicting TIEG gene expression in primary breast cancer tumors from node negative (N−) good outcome patients and N− bad outcome patients. Average TIEG gene expression is shown as a percentage of TIEG expression in normal breast tissue. SEMs are presented as the error bars. *: p≦0.004 by Student's t-test, p<0.0002 by Wilcoxon Rank-Sum test versus normal. Sample size for each group is shown in parentheses. FIG. 4B is a bar graph depicting individual tumor TIEG gene expression of N− good and bad primary breast cancers. TIEG gene copy number is shown as TIEG gene copy number in normal breast tissue number×1000/pg β-actin mNRA. With regard to both FIGS. 4A and 4B, good outcome patients experience no disease recurrence at 5 years after initial diagnosis, and bad outcome patients experience a disease recurrence at less than 3 years after initial diagnosis.

FIG. 5A is a bar graph depicting TIEG gene expression in normal, ductal carcinoma in situ (DCIS), primary tumor N−/node positive (N+), N− good outcome, and N− bad outcome tissues. Average TIEG gene expression, as assessed with the TIEG80 primers described herein, is shown in units of gene copies×1000/pg β-actin mRNA. SEMs are presented as the error bars. Sample sizes are indicated below each tissue type. FIG. 5B is a bar graph depicting TIEG gene expression in normal, DCIS, primary tumor N−/N+, metastatic liver, and metastatic ovary tissues. Average TIEG gene expression, as assessed with the TIEG80 primers described herein, is shown in units of gene copies×1000/pg β-actin mRNA. SEMs are presented as the error bars. Sample sizes are indicated below each tissue type.

FIG. 6A is a bar graph depicting TIEG gene expression in normal, DCIS, primary tumor N−/N+, N− good outcome, and N− bad outcome tissues corrected for epithelial cell content. Average TIEG gene expression, as assessed with the TIEG80 primers described herein, is shown in units of gene copies×1000/pg β-actin mRNA. SEMs are presented as the error bars. Sample sizes are indicated below each tissue type. FIG. 6B is a bar graph depicting TIEG gene expression in normal, DCIS, primary tumor N−/N+, metastatic liver, and metastatic ovary tissues corrected for epithelial cell content. Average TIEG gene expression, as assessed with the TIEG80 primers described herein, is shown in units of gene copies×1000/pg β-actin mRNA. SEMs are presented as the error bars. Sample sizes are indicated below each tissue type.

FIG. 7A is a bar graph depicting BARD-1 gene expression in normal, DCIS, primary tumor N−/N+, N− good outcome, and N− bad outcome tissues. Average BARD-1 gene expression is shown in units of gene copies×1000/pg β-actin mRNA. SEMs are presented as the error bars. Sample sizes are indicated below each tissue type. FIG. 7B is a bar graph depicting BARD-1 gene expression in normal, DCIS, primary tumor N−/N+, metastatic liver, metastatic femur, and metastatic ovary tissues. Average BARD-1 gene expression is shown in units of gene copies×1000/pg β-actin mRNA. SEMs are presented as the error bars. Sample sizes are indicated below each tissue type.

FIG. 8A is a bar graph depicting Smad 7 gene expression in normal, DCIS, primary tumor N−/N+, N− good outcome, and N− bad outcome tissues. Average Smad 7 gene expression is shown in units of gene copies×1000/pg β-actin mRNA. SEMs are presented as the error bars. Sample sizes are indicated below each tissue type. FIG. 8B is a bar graph depicting Smad 7 gene expression in normal, DCIS, primary tumor N−/N+, metastatic liver, metastatic femur, and metastatic ovary tissues. Average Smad 7 gene expression is shown in units of gene copies×1000/pg β-actin mRNA. SEMs are presented as the error bars. Sample sizes are indicated below each tissue type.

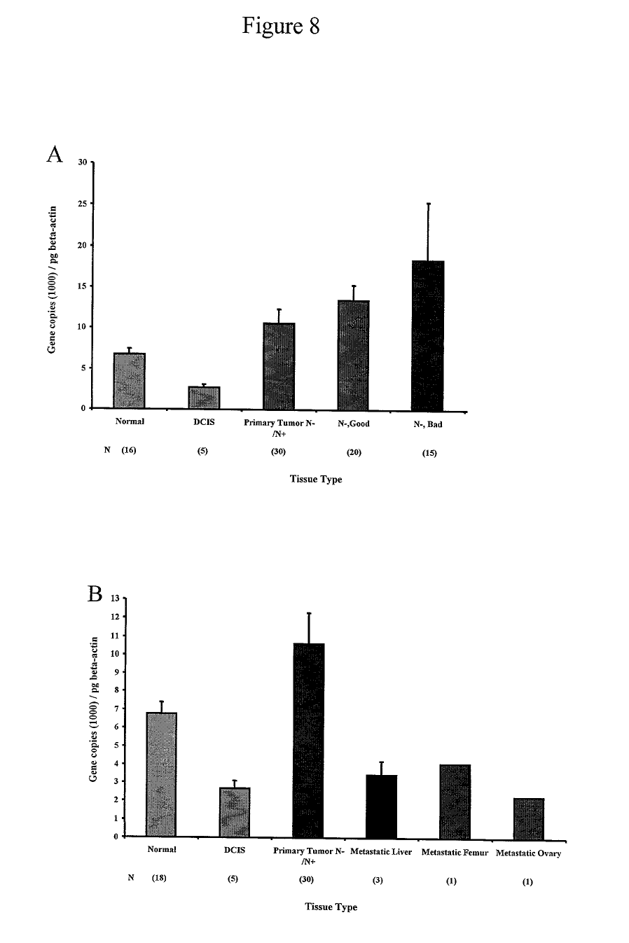

FIG. 9A is a bar graph depicting Smad 2 gene expression in normal, DCIS, primary tumor N−/N+, N− good outcome, and N− bad outcome tissues. Average Smad 2 gene expression is shown in units of gene copies×1000/pg β-actin mRNA. SEMs are presented as the error bars. Sample sizes are indicated below each tissue type. FIG. 9B is a bar graph depicting Smad 2 gene expression in normal, DCIS, primary tumor N−/N+, metastatic liver, metastatic femur, and metastatic ovary tissues. Average Smad 2 gene expression is shown in units of gene copies×1000/pg β-actin mRNA. SEMs are presented as the error bars. Sample sizes are indicated below each tissue type.

### DETAILED DESCRIPTION

In general, the invention provides methods for determining the aggressiveness of a cancer by determining the level of a biomolecule in a test sample from a mammal. The level of a biomolecule can be correlated with the aggressiveness of a cancer. Identifying aggressive cancers at an early stage can help a physician properly diagnose and treat a cancer patient. Typically, a properly diagnosed and treated cancer patient can experience an improvement in general health and survival.

The term “aggressive” as used herein refers to the metastatic potential of a cancer. Metastatic potential refers to the tendency of cancer cells to move from one part of the body to another. This can lead to formation of secondary growths at new locations distant from the primary, or original, tumor. The cancer cells can spread to other locations via the bloodstream, the lymphatic system, or by other routes such as the cerebrospinal fluid. Metastatic potential of malignant breast tumors, for example, can be detected by examining nearby or distant lymph nodes for the present of cancer cells. An aggressive cancer can produce adverse changes in a mammal's overall health to a greater extent than if that cancer were not aggressive. A mammal with an aggressive cancer can, for example, experience pain associated with metastasis, enlargement and dysfunction of organs such as lymph nodes, lungs, and liver, pathological bone fractures, loss of appetite, mineral and vitamin deficiencies, increased risk of infection, and depression to a greater extent than if that cancer were not aggressive. Aggressive cancers can increase mortality to a greater extent than cancers that are not aggressive. Various types of cancer can be aggressive, including, without limitation, solid tissue cancers such as breast, prostate, skin (e.g., melanoma), brain, colon, lung, ovary, and bladder.

The term “biomolecule” as used herein refers to DNA, RNA, or polypeptides. The invention provides methods for measuring biomolecules related to, without limitation, signaling factors such as the receptor-regulated Smads (e.g., Smad 2 and Smad 3), inhibitory Smads (e.g., Smad 6 and Smad 7) and common mediator Smads (e.g., Smad 4), transcription factors such as the TGF-β inducible early genes (TIEGs), and other factors such as BRCA1-associated RING domain (BARD).

The term “marker” as used herein refers to a test level of a biomolecule that is either altered or normal compared to a control level. The level of a particular biomolecule can be measured in a test sample from a mammal. The resulting test level then can be compared to a control level of the corresponding biomolecule. If a test level is altered compared to a control level, then the cancer in the mammal corresponding to that test sample can be classified as aggressive. For example, if the level of TIEG mRNA measured in a breast cancer sample is reduced compared to a control level of TIEG mRNA, then that breast cancer can be classified as aggressive. In another example, if the level of Smad 7 mRNA measured in a breast cancer sample is elevated compared to a control level of Smad 7 mRNA, then that breast cancer can be classified as aggressive. Alternatively, if a test level is normal compare to a control level, then the cancer corresponding to that test sample can be classified as not aggressive. For example, if the level of Smad 7 mRNA measured in a breast cancer sample is normal compared to a control level of Smad 7 mRNA, then that breast cancer can be classified as not aggressive. Markers can be referred to in groups that reflect a common characteristic or use. For example, altered or normal levels of Smad 7 and TIEG mRNA are referred to as TIEG markers because both Smad 7 and TIEG mNRA levels can be used to determine whether or not a mammal has cancer, or whether or not a mammal diagnosed with cancer has an aggressive cancer. Other non-limiting examples of TIEG markers include levels of Smad 2, Smad 3, Smad 4, and BARD-1 biomolecules.

Suitable control levels include, without limitation, an average level of a particular biomolecule from mammals without cancer, an average level of a particular biomolecule from mammals with a benign tumor, or a level of a particular biomolecule from a non-cancerous tissue or organ (e.g., breast, ovary, testis, lung, or kidney) from the same or a different mammal. Alternatively, if the level of a particular biomolecule associated with a particular cancer has been characterized such that a standard control level or baseline is known, comparing a test level to the corresponding baseline for that particular cancer can identify an aggressive cancer. For example, if the level of TIEG mRNA measured in a breast cancer sample is reduced compared to a baseline of TIEG mRNA for breast cancer, then that breast cancer in that mammal can be classified as aggressive.

As used herein, “baseline” typically refers to a defined range of levels established from studies of multiple normal and cancer patients. In typical practice, the upper limit of a baseline defines one cutoff (e.g., for either normal or cancer conditions) and the lower limit of the baseline defines another cutoff (e.g., for either cancer or normal conditions) of the diagnostic or prognostic spectrum. For example, in a hypothetical situation where measurements of TIEG range from 1 to 10 gene copies (i.e., 1 to 10 mRNA molecules)×1000 per pg β-actin mRNA, it might be determined that values of 7 or above indicate a patient is cancer free, and values of 4 or below indicate a patient has cancer. Patients with values of 5 or 6 would be in need of further testing, because the previously established baseline values of 7 (upper) and 4 (lower) do not provide unambiguous results for values between 7 and 4. In the above hypothetical situation, the “baseline” is the defined range of 4 to 7 gene copies×1000 per pg β-actin mRNA. Baselines can be adjusted to establish optimum levels of specificity and sensitivity for particular diagnostic or prognostic formats and patient populations.

Levels of the same type of biomolecule typically are used when comparing a test level to a control level (e.g., an mRNA test level to an mRNA baseline, or a polypeptide test level to a polypeptide control level). Further, when comparing a test level to a control level or baseline, a statistically significant difference can indicate that the test level is altered compared to the control level or baseline. For example, a Smad 7 mNRA level can be considered elevated compared to a corresponding baseline when that level is statistically significantly greater than the corresponding baseline. In addition, if the difference between a test level and a control level or baseline is not statistically significant, then that test level can be considered normal compared to the control level or baseline. Typically, a difference in levels is considered statistically significant at p≦0.05 with an appropriate parametric or non-parametric statistic, e.g., Chi-square test, Student's t-test, Mann-Whitney test, Wilcoxon Rank-Sum test, or F-test. In some embodiments, a difference in levels is statistically significant at p<0.01, p<0.005, or p <0.001.

The presence of a marker in a test sample can be correlated with the aggressiveness of a cancer. For example, the presence of a reduced level of TIEG mRNA in a breast cancer sample indicates that that breast cancer is aggressive. In another example, the presence of an elevated level of Smad 7 mRNA in a breast cancer sample indicates that that breast cancer is aggressive. Alternatively, the absence of a marker also can be correlated with the aggressiveness of a cancer. For example, the absence of a reduced level of TIEG mRNA in a breast cancer sample from a mammal indicates that that breast cancer is not aggressive. Further, the presence of a normal level of Smad 7 mRNA in a breast cancer sample indicates that that breast cancer is not aggressive.

In another embodiment, the presence or absence of multiple markers can be used to determine the aggressiveness of a cancer. In general, it is useful to measure TIEG biomolecule levels in combination with levels of any other biomolecules regulated by TIEG, although combinations of TIEG markers that do not include TIEG levels are also useful in some circumstances. For example, the presence of a reduced level of TIEG mNRA and an elevated level of Smad 7 mRNA in a breast cancer sample from a mammal indicates that that breast cancer in that mammal is aggressive. Other non-limiting examples of suitable combinations of markers include TIEG biomolecule levels in combination with any biomolecule level of Smad 2, Smad 7, and BARD-1; Smad 2 biomolecule levels in combination with any biomolecule level of TIEG, Smad 7, and BARD-1; Smad 7 biomolecule levels in combination with any biomolecule level of Smad 2, TIEG, and BARD-1; and BARD-1 biomolecule levels in combination with any biomolecule level of Smad 2, Smad 7, and TIEG. Assessing the presence or absence of multiple markers can improve the accuracy of determining cancer aggressiveness. When using the presence or absence of multiple markers to determine cancer aggressiveness, the markers can be from the same or different marker groups. For example, the presence or absence of a TIEG marker and a p53 marker (e.g., a level of a biomolecule that is structurally or functionally related to p53) can be used to determine cancer aggressiveness.

The presence or absence of markers can be particularly useful in predicting a prognosis of a cancer patient. A “good outcome” prognosis refers to no disease recurrence at 5 years after initial diagnosis. A “bad outcome” prognosis refers to a disease recurrence at less than 3 years after initial diagnosis. For example, the presence of an elevated level of Smad 7 mRNA in a breast cancer sample from a patient can be used to predict a bad outcome for that patient. This prognostic value of the present invention can be useful to a physician in designing appropriate therapeutic regimens.

Various types of samples can be used when measuring a biomolecule level. Such samples include, without limitation, tissue biopsies, surgical waste, isolated cells (e.g., captured epithelial cells from a blood sample), and whole organs. Cancer biopsy specimens can be frozen, embedded, sectioned, and stained to identify cancerous regions.

Methods for Measuring a Biomolecule Level

Various methods can be used to measure a biomolecule level in a sample. Such methods can vary depending on the type of biomolecule measured. Methods for measuring RNA levels include, without limitation, hybridization (e.g., Northern blotting of separated RNAs, and dot or slot blotting or total RNA) and PCR-based methods (e.g., RT-PCR and quantitative real-time PCR). For example, hybridization can be done by Northern analysis to identify an RNA sequence that hybridizes to a probe. The probe can be labeled with a radioisotope such as ³²p, an enzyme, digoxygenin, or by biotinylation. The RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring Harbor Laboratory, Plainview, N.Y.

As standard Northern blot assays can be used to ascertain the level of a particular RNA in a sample from a mammal, so can PCR-based methods such as quantitative real-time PCR. In one embodiment, reverse transcription using random hexamer oligonucleotide primers can be performed on total mRNA isolated from a cancer sample. The resulting cDNA then can be used as template in quantitative real-time PCR experiments using forward and reverse oligonucleotide primers in the presence of a specific probe (e.g., a probe having a 5′ fluorescent reporter dye at one end and a 3′ quencher dye at the other end). Reactions can be monitored using the point during cycling when amplification of a PCR product is first detected, rather than the amount of PCR product accumulated after a fixed number of cycles. The resulting quantitated PCR product levels can be correlated to the mRNA levels in the original cancer sample, and the mRNA levels can in turn be correlated with the aggressiveness of that cancer.

Methods for measuring polypeptide levels include, without limitation, ELISA-, immunohistochemistry-, and immunofluorescence-based techniques. Such methods typically employ antibodies having specific binding affinity for a particular polypeptide. “Specific binding affinity” refers to an antibody's ability to interact specifically with a particular polypeptide without significantly cross-reacting with other different polypeptides in the same environment. An antibody having specific binding affinity for TIEG can interact with TIEG polypeptides specifically in the presence of multiple different polypeptides, for example, multiple different Smads. TIEG antibodies can have specific binding affinity for full-length or fragments of TIEG from any suitable species, including, without limitation, mouse, rat, chimpanzee, and human. For example, TIEG antibodies can have specific binding affinity for a full-length human TIEG polypeptide (SEQ ID NO:25) or fragments of a human TIEG polypeptide including, without limitation, amino acids 20-39 (Ser-Glu-Arg-Pro-Lys-Glu-Ser-Met-Tyr-Ser-Trp-Asn-Lys-Thr-Ala-Glu-Lys-Ser-Asp-Phe; SEQ ID NO:26), amino acids 94-106 (Pro-Pro-Tyr-Ser-Pro-Ser-Asp-Phe-Glu-Pro-Ser-Gln-Val; SEQ ID NO:27), amino acids 134-154 (Phe-Lys-Glu-Glu-Glu-Lys-Ser-Pro-Val-Ser-Ala-Pro-Lys-Leu-Pro-Lys-Ala-Gln-Ala-Thr-Ser; SEQ ID NO:28), and amino acids 353-370 (Ser-Ala-Ala-Lys-Val-Thr-Pro-Gln-Ile-Asp-Ser-Ser-Arg-Ile-Arg-Ser-His-Ile; SEQ ID NO:29).

TIEG polypeptide levels in a breast cancer sample can, for example, be measured using a quantitative sandwich ELISA technique. Breast cancer tissue samples can be homogenized and extracted, and aliquots of the extracts added to separate wells of a microtiter plate pre-coated with antibodies specific for TIEG. After protein binding and subsequent washing, enzyme-linked antibodies specific for TIEG can be added to the wells. After antibody binding and subsequent washing, a substrate solution containing a label-conjugated IgG can be added to the wells (e.g., horseradish peroxidase (HRP)-conjugated IgG). The label then can be quantitated by spectrophotometry and the quantitated levels compared to a control level or baseline. The resulting quantitated polypeptide levels can be correlated with the aggressiveness of that cancer.

Polypeptide levels also can be measured by immunohistochemistry. For example, a section of a breast cancer tissue sample can be treated with anti-BARD-1 primary antibodies, while an adjacent section from the same sample can be treated with anti-TIEG primary antibodies. Negative control sections can be incubated with pre-immune rabbit or mouse serum in lieu of primary antibodies. After antibody binding and subsequent washing, the primary antibodies can be detected with appropriate label-conjugated secondary antibodies (e.g., gold-conjugated or enzyme-conjugated antibodies). The label is then developed and quantitated using an image analysis system. The resulting quantitated polypeptide levels can be correlated with the aggressiveness of that cancer. Although samples can be processed individually, samples from different tissues or from a population of different patients can be processed simultaneously. Such processing methods include, without limitation, tissue microarrays, as described by Kononen et al. (Nat. Med., 4:844-847, 1998).

Immunofluorescence techniques represent another approach to measuring the level of a polypeptide. For example, BARD-1 and TIEG polypeptides can be localized in the same breast cancer sample section using polyclonal and monoclonal antibodies against BARD-1 and TIEG. The bound antibodies are detected using different fluorescently conjugated antibodies. The levels of BARD-1 and TIEG fluorescence are quantitated using an image analysis system, and the resulting quantitated levels correlated with the aggressiveness of that cancer.

Suitable antibodies for ELISA-, immunohistochemistry- and immunofluorescence-based methods can be obtained using standard techniques. In addition, commercially available antibodies to polypeptides associated with cancer aggressiveness can be used.

Kits

The invention provides kits that can be used to determine the level of a TIEG nucleic acid in a sample. The term “TIEG nucleic acid” refers to any nucleic acid encoding a full or partial TIEG polypeptide. Kits can contain an oligonucleotide primer pair that specifically amplifies all or a portion of a target region of a TIEG nucleic acid. Target regions can be defined at any place along a TIEG nucleic acid. For example, a target region can be defined by nucleotides 1-500 of the 5′ portion of a TIEG nucleic acid. In this case, a kit of the invention can contain an oligonucleotide primer pair that specifically amplifies all 500 nucleotides defining that target region, or a portion (e.g., nucleotides 80-188) of that target region. The numbering of nucleotides in a TIEG nucleic acid follows conventional numbering practices. Nucleotide positions that are 3′ of the “A” in a TIEG initiation codon are designated as “X” or “+X” relative to the “A” in the initiation codon. Nucleotide positions that are 5′ of the “A” in a TIEG initiation codon are designated as “−X” relative to the “A” in the initiation codon. In both cases, the “A” in the initiation codon is given the number “1” or “+1”.

The invention further provides kits that can be used to determine the level of a TIEG marker-related nucleic acid in a sample. As used herein, the term “TIEG marker-related nucleic acid” refers to any nucleic acid encoding a full or partial polypeptide corresponding to any TIEG marker described herein. Again, such markers include, without limitation, levels of TIEG, BARD-1, Smad 7, and Smad 2 biomolecules. Thus, any nucleic acid encoding a full or partial polypeptide corresponding to, for example, BARD-1 is a TIEG marker-related nucleic acid.

Components and methods for producing kits are well known. Kits can contain multiple oligonucleotide primer pairs that specifically amplify TIEG marker-related nucleic acids, or probes that specifically hybridize to TIEG marker-related nucleic acids. In addition, kits can contain antibodies for detecting TIEG marker-related polypeptides. The kits provided herein also can contain a reference chart that indicates a reference level or baseline for TIEG marker-related polypeptides or nucleic acids. Kits can be configured in any type of design (e.g., microtiter plate design) and can be made of any type of material (e.g., plastic).

It is understood that the term “specifically amplifies” refers to the ability of an oligonucleotide primer to interact specifically with a particular nucleic acid without significantly cross-reacting with other different nucleic acids in the same environment and facilitate or promote the amplification of that particular nucleic acid. Likewise, the term “specifically hybridizes” refers to the ability of an oligonucleotide probe to interact specifically with a particular nucleic acid without significantly cross-reacting with other different nucleic acids in the same environment and facilitate or promote the detection of that particular nucleic acid.

Methods for Assisting a Person in Determining the Aggressiveness of a Cancer

The invention also provides methods to assist a person in determining the aggressiveness of a cancer in a mammal. Such a person can be, for example, a physician, a nurse, a medical laboratory technologist, or a pharmacist. A person can be assisted by (1) determining the presence or absence of a TIEG marker in a test sample, and (2) communicating information about the presence or absence of that marker to that person.

Any method can be used to communicate information to another person. For example, information can be given directly or indirectly to a person. In addition, any type of communication can be used to communicate the information. For example, mail, e-mail, telephone, and face-to-face interactions can be used. The information also can be communicated to a person by making that information electronically available to the person. For example, the information can be communicated to a person by placing the information on a computer database such that the person can access the information. In addition, the information can be communicated to a hospital, clinic, or research facility at which the person is located.

The invention will be further described in the following examples, which do not limit the scope of the invention described in the claims.

### EXAMPLES

### Example 1

Regulation of TIEG Markers

Cells from the Hs578T cell line (ATCC No: HTB 126) were made to inducibly express TIEG in the presence of doxycycline using the T-Rex system (Invitrogen, Carlsbad, Calif.) according to the manufacturer's instructions. The resulting cell line was referred to as Hs578T-TIEG. Separate dishes of Hs578T-TIEG cells were cultured in DMEM/F12 (1:1) medium containing 10% (v/v) fetal bovine serum (BioWhittaker, Walkersville, Md.), 5 mg/L blasticidin (Invitrogen), 500 mg/L Zeocin® (Invitrogen), and 1× antibiotic-antimycotic solution (Life Technologies, Rockville, Md.). Some dishes of cells were designated as containing test cells, and others were designated as containing control cells. The test cells received 50 ng/mL doxycycline (Sigma, St. Louis, Mo.), and both the test cells and the control cells were incubated at 37° C., 5% CO₂. After 24 hours, total RNA from both groups of cells was isolated using Tri-Reagent phenol-guanidine isothiocyanate solution (Molecular Research Center, Cincinnati, Ohio) to give test RNA and control RNA. See, Subramaniam, M., et al., Nucleic Acids Res., 23:4907-12 (1995).

Test RNA and control RNA (10 μg each) were separated electrophoretically in a tris-acetate 1% (w/v) agarose gel. The resulting separated test and control RNA was then electrophoretically transferred in 20×SSC to a nylon membrane. After 24 hours, the amount of Smad 7 mRNA in both test and control RNA samples was determined by Northern blotting using the nylon membrane containing the transferred RNA. Briefly, the membrane was treated with a 236 bp [α³²P]-dCTP-labeled fragment of a human Smad 7 cDNA (specific activity >10⁸ cpm/μg). This probe hybridizes to human or mouse Smad 7. Hybridization was in 25 ml deionized formamide, 5 ml 50× Denhardt's solution, 7.5 ml 20×SSC, 250 μl Poly A (2 mg/ml), 500 μl 10% SDS, 500 μl sheared salmon sperm DNA (5 mg/ml), made up to 50 ml with sterile water. Hybridization was for at least 22 hrs at 43° C. After hybridization and subsequent washing to remove unhybridized probe, the membrane was then exposed to film at −70° C. After 18 hours, bands on the exposed film representing Smad 7 mRNA from both the test and control cells were quantitated by densitometry.

To determine whether TIEG regulates Smad 7 transcription, AKR2B cells (an AKR2B mouse fibroblast cell line) were transfected with (1) a TIEG expression construct, (2) a reporter construct including a firefly luciferase gene driven by portions of either a human (bases −2000 to +672) or mouse (bases −408 to +112) Smad 7 promoter, and (3) a reporter construct including a renilla luciferase gene. Cell extracts from the transfected cells were harvested 48 hours after transfection using 1× passive lysis buffer (Promega, Madison, Wis.). Luciferase assays were performed on the cell extracts using the Dual-Luciferase™ Reporter Assay System (Promega) as described by the manufacturer, and both firefly and renilla luciferase units were measured using a luminometer (TD-20/20; Turner Designs, Sunnyvale, Calif.). To correct for transfection efficiency, firefly luciferase units were normalized to renilla luciferase within each cell extract.

Hs578T-TIEG test cells treated with doxycycline, and therefore overexpressing TIEG, exhibited a reduced level of endogenous Smad 7 mRNA compared to the level of Smad 7 mRNA in control cells. Smad 7 promoter assays performed on cell extracts from transfected and control AKR2B cells revealed that TIEG repressed mouse Smad 7 promoter activity by 80%. TIEG also repressed the human Smad7 promoter by 84%, but had no effect on an unrelated (cyclin D1) promoter. Similar results were obtained in Hs578T, MDA-MB-231, SK-BR-3 and Mv1Lu cell lines expressing TIEG.

These data demonstrate that TIEG downregulates endogenous Smad 7 gene expression. These data also demonstrate that TIEG regulates the expression of other TEIG marker-related nucleic acids.

### Example 2

Predicting Patient Outcome Using TIEG Markers

Breast tissue samples were obtained from the following sources: 16 normal breast tissue samples from both pre-and post-menopausal women between 26 and 75 years of age; 69 primary breast tumor tissue samples from patients without signs of distant metastasis at the time of surgery and between 33 and 86 years of age, including 13 node-positive (N+) and 17 node-negative (N−) samples with no correlative patient outcome data, 20 N− samples correlated with good patient outcome, and 14 N− samples correlated with bad patient outcome; 5 ductal carcinoma in situ (DCIS) samples; and 5 distant metastases (3 liver, 1 ovary, and 1 femur) samples. Good patient outcome was defined as no disease recurrence at 5 years after initial diagnosis, and bad patient outcome was defined as disease recurrence at less than 3 years after initial diagnosis. All tissue samples were processed in a pathology frozen section laboratory, snap frozen, and stored at −80° C. until used. The histology, tumor size, nodal status, Her2 status, estrogen receptor (ER) status, and progesterone receptor (PR) status of each tissue sample was determined (see Tables 1 and 2). In addition, the 69 primary tumor samples were staged according to the TNM classification system. See, Fitzgibbons, P. L., et al., Arch. Pathol. Lab. Med., 124:1026-1033 (2000).

TABLE 1
Pathological characteristics of 30 N+/N− breast cancer patients.

| Patient # | Histology | Tumor Size | Nodal status | Her2 status | ER status | PR status |
|---|---|---|---|---|---|---|
| 1 | ID | T1 | − | − | + | + |
| 2 | ID | T1 | − | − | + | + |
| 3 | IL | T1 | − | − | + | + |
| 4 | ID | T1 | − | 2+ | + | + |
| 5 | ID | T1 | − | 3+ | + | + |
| 6 | ID | T1 | − | − | + | + |
| 7 | ID | T1 | − | − | + | + |
| 8 | IL | T3 | − | − | + | + |
| 9 | IS | T1b | − | − | − | − |
| 10 | ID | T2 | − | − | − | − |
| 11 | ID | T3 | − | − | − | + |
| 12 | ID | T1c | − | 3+ | + | + |
| 13 | IL | T2 | − | − | + | + |
| 14 | ID | T2 | − | − | − | − |
| 15 | ID | T1c | − | − | + | + |
| 16 | ID | T1c | − | 3+ | + | + |
| 17 | ID | T2 | − | 3+ | + | + |
| 18 | IL | T1c | N2 | − | + | + |
| 19 | IL | T2 | N1b | − | + | + |
| 20 | ID | T2 | N1b | − | + | + |
| 21 | ID | T2 | N1b | 2+ | + | + |
| 22 | ID | T3 | N1b | − | + | + |
| 23 | ID | T2 | N1b | − | + | + |
| 24 | ID | T2 | N1b | 3+ | − | + |
| 25 | IL | T2 | N1b | 3+ | + | − |
| 26 | ID | T2 | N1b | 2+ | + | + |
| 27 | ID | T2 | N1b | 3+ | + | + |
| 28 | ID | T3 | N1b | 3+ | − | + |
| 29 | ID | T2 | N1a | − | + | + |
| 30 | ID | T2 | N1 | 3+ | − | − |
| Positive nodal status (+) is determined as having at least one positive axillary node detected at the time of primary surgery. Positive Her2 status (+) is determined as having a 2+ or 3+ by immunohistochemical staining using HercepTest ™. A positive ER or PR status (+) is determined as having any neoplastic cell immunohistochemically stained for ER or PR. IL: invasive lobular carcinoma; ID: invasive ductal carcinoma; IS: invasive syringomatous carcinoma; | | | | | | |
| # ER estrogen receptor; PR: progeterone receptor; T1: tumor 2 cm or less in greatest dimension; T1b: tumor >0.5 cm but not >1 cm in greatest dimension; T1c: tumor >1 cm but not >2 cm in greatest dimension; T2: tumor >2 cm but not >5 cm in greatest dimension; T3: tumor >5 cm in greatest dimension; N1: metastasis to movable ipsilateral axillary lymph node(s); N1a: only micrometastasis (none >0.2 cm in greatest dimension); N1b: metastasis to lymph node(s), any | | | | | | |
| # >0.2 cm in greatest dimension; N2: metastasis to ipsilateral axillary lymph node(s) fixed to one another or to other structures. | | | | | | |

TABLE 2
Pathological characteristics and outcome of 35 N− breast cancer patients.

| Patient # | Outcome | Histology | Tumor Size | Her2 status | ER status | PR status |
|---|---|---|---|---|---|---|
| 1 | Good | ID | T1 | − | + | + |
| 2 | Good | ID | T1 | − | + | + |
| 3 | Good | ID | T1 | 2+ | + | + |
| 4 | Good | ID | T2 | 2+ | + | + |
| 5 | Good | ID | T2 | − | + | + |
| 6 | Good | ID | T1 | − | + | + |
| 7 | Good | ID | T1 | − | + | + |
| 8 | Good | ID | T2 | − | + | + |
| 9 | Good | ID | T2 | 2+ | + | + |
| 10 | Good | ID | T2 | − | + | + |
| 11 | Good | ID | T2 | − | − | + |
| 12 | Good | IL | T2 | − | + | + |
| 13 | Good | ID | T2 | − | − | − |
| 14 | Good | ID | T2 | − | − | − |
| 15 | Good | ID | T3 | − | + | + |
| 16 | Good | ID | T2 | − | + | + |
| 17 | Good | IL | T3 | − | + | + |
| 18 | Good | ID | T2 | − | + | + |
| 19 | Good | ID | T2 | − | + | + |
| 20 | Good | ID | T3 | − | + | + |
| 21 | Bad | ID | T1 | − | + | + |
| 22 | Bad | ID | T1 | − | − | − |
| 23 | Bad | IL | T1 | 3+ | + | + |
| 24 | Bad | ID | T2 | 3+ | − | + |
| 25 | Bad | ID | T1 | − | + | − |
| 26 | Bad | ID | T2 | − | − | − |
| 27 | Bad | ID | T3 | 3+ | + | − |
| 28 | Bad | ID | T2 | 3+ | − | − |
| 29 | Bad | ID | T2 | − | − | − |
| 30 | Bad | ID | T1 | 3+ | − | − |
| 31 | Bad | ID | T3 | − | + | + |
| 33 | Bad | ID | T2 | − | − | − |
| 34 | Bad | ID | T2 | − | + | + |
| 35 | Bad | ID | T2 | − | − | + |
| Positive Her2 status (+) is determined as having 2+ or 3+ by immunohistochemical staining using HercepTest ™. A positive ER or PR status (+) is determined as having any neoplastic cell immunohistochemically stained for ER or PR. IL: invasive lobular carcinoma; ID: invasive ductal carcinoma; IS: invasive syringomatous carcinoma; ER: estrogen receptor; PR: progesterone receptor; T1: tumor 2 cm or less in greatest dimension; | | | | | | |
| # T2: tumor >2 cm but not >5 cm in greatest dimension; T3: tumor >5 cm in greatest dimension. | | | | | | |

The frozen samples were cut into 20 μ thick sections using a cryostat, and 10 sections from each sample were placed in sterile microfuge tubes. Total RNA was isolated from the sections using 1 mL Trizol® reagent (Gibco BRL, Grand Island, N.Y.) according to the manufacturer's instructions. The isolated RNA was extracted with chloroform and precipitated with isopropyl alcohol. The precipitated RNA was pelleted by centrifugation, and the resulting pellet washed with 75% ethanol. After aspirating the ethanol, the pellet was allowed to dry. The dried pellet was then dissolved in 50 μL DEPC-treated water (RNase/DNase free). A 50-μL aliquot of the dissolved RNA was then incubated with 1 U RQ1 RNase-free DNase I (Promega) at 37° C. for 30 minutes. The DNase I was removed using a kit (RNeasy®; Qiagen, Hilden, Germany) to give purified total RNA. 1 μg of the purified total RNA was used as template in a reverse transcription (RT) reaction with random hexamer primers (Roche Biopharmaceuticals, Indianapolis, Ind.). The RT reaction mixture, including template, hexamer primers, AMV reverse transcriptase, nucleotides, and MgCl₂ buffer in a total volume of 20 μL, was incubated at 37° C. for 60 minutes to give cDNA.

The resulting cDNA was used as template in quantitative real-time PCR experiments. See, Holland et al., Proc. Natl. Acad. Sci. USA, 88:7276-7280 (1991); Heid, C. A., et al., Genome Res., 6:986-994 (1996). cDNA template was amplified by PCR using forward and reverse oligonucleotide primers in the presence of a specific probe having a 5′ fluorescent reporter dye at one end and a 3′ quencher dye at the other end. Reactions were monitored using the point during cycling when amplification of a PCR product was first detected, rather than the amount of PCR product accumulated after a fixed number of cycles. Thus, the larger the starting quantity of a particular RNA used in the RT reaction to generate template cDNA, the earlier the observed increase in fluorescence. The amount of RNA in unknown samples was quantified by measuring Ct (defined as the fractional cycle number at which the fluorescence generated by cleavage of the probe passes a fixed threshold above baseline) and by using a standard curve. cDNA amplifications and real-time fluorescence quantitations were performed using an ABI 7700 Prism sequence detection system (Applied Biosystems, Foster City, Calif.).

Matching primers and probes were designed in different regions of TIEG, Smad 7, Smad 2 and BARD-1 using Primer Express software (version 1.5; Applied Biosystems). TIEG primers and probes were based upon human sequence data for TIEG (Subramaniam et al., Nucleic Acids Res., 23:4907-4912 (1995); Genbank accession # U21847). Smad 7 primers and probes were based upon Genbank accession # AF010193. Smad 2 primers and probes were based upon Genbank accession # NM 005901. BARD-1 primers and probes were based upon Genbank accession # XM 00236412. The sequences and positions of the primers and the probe for each reaction are shown in Table 3 and are designated TIEG80 (amplifies/recognizes a gene region encoding a far N-terminal region), TIEG515 (amplifies/recognizes a gene region encoding a mid N-terminal region), TIEG529 (amplifies/recognizes a gene region encoding a mid-N-terminal region), TIEG1217 (amplifies/recognizes a gene region encoding a C-terminal zinc-finger region), Smad 7 (amplifies/recognizes a gene region encoding amino acids 180-202 of the N-terminal region), Smad 2 (amplifies/recognizes a gene region encoding amino acids 103-132 of the N-terminal region), and BARD-1 (amplifies/recognizes a gene region encoding amino acids 362-565 of the C-terminal region).

TABLE 3
Primer and probe sequences

| | | | | Product |
|---|---|---|---|---|
| | | | | Size |
| Gene | Sequence | | Position | (BP) |
| β-actin | | | | 295 |
| Forward Primer | TCACCCACACTGTGCCCATCTACGA | (SEQ ID NO:1) | 2141 | |
| Reverse Primer | CAGCGGAACCGCTCATTGCCAATGG | (SEQ ID NO:2) | 2411 | |
| Probe | ATGCCCCCCCCATGCCATCCTGCGT | (SEQ ID NO:3) | 2171 | |
| TIEG80 | | | | 108 |
| Forward Primer | GCCAACCATGCTCAACTTCG | (SEQ ID NO:4) | 80 | |
| Reverse Primer | TGCAGTTTTGTTCCAGGAATACAT | (SEQ ID NO:5) | 188 | |
| Probe | TGCCTCTCTCCAGCAGACTGCGGA | (SEQ ID NO:6) | 101 | |
| TIEG515 | | | | 67 |
| Forward Primer | TGCCCCCAAACTCCCC | (SEQ ID NO:7) | 515 | |
| Reverse Primer | ACATAGCTGGGCATCAGCTGT | (SEQ ID NO:8) | 581 | |
| Probe | AAGCTCAGGCAAGTGTGATTCGTCA | (SEQ ID NO:9) | 532 | |
| TIEG529 | | | | 72 |
| Forward Primer | CCAAAGCTCAGGCAACAAGTG | (SEQ ID NO:10) | 529 | |
| Reverse Primer | TTGGGCAGGTCTGGTGGTTA | (SEQ ID NO:11) | 600 | |
| Probe | ATTCGTCATACAGCTGATGCCCAGCTATG | (SEQ ID NO:12) | 552 | |
| TIEG1217 | | | | 93 |
| Forward Primer | CAAGACATACTTTAAAAGTTCCCATCTG | (SEQ ID NO:13) | 1217 | |
| Reverse Primer | CTTTCACAACCTTTCCAGCTACAG | (SEQ ID NO:14) | 1309 | |
| Probe | AGGCCCACACGAGGACGCACA | (SEQ ID NO:15) | 1246 | |
| Smad 7 | | | | 67 |
| Forward Primer | GAATCTTACGGGAAGATCAACCC | (SEQ ID NO:16) | 836 | _____ |
| Reverse Primer | CGCAGAGTCGGCTAAGGTG | (SEQ ID NO:17) | 902 | _____ |
| Probe | AGCTGGTGTGCTGCAACCCCA | (SEQ ID NO:18) | 861 | |
| Smad 2 | | | | 86 |
| Forward Primer | AGAGAGTTGAGACACCAGTTTTGC | (SEQ ID NO:19) | 558 | _____ |
| Reverse Primer | ATAGTCATCCAGAGGCGGAAGTT | (SEQ ID NO:20) | 643 | ______ |
| Probe | AGTGCCCCGACACACCGAGATCCT | (SEQ ID NO:21) | 592 | |
| BARD-1 | | | | 107 |
| Forward Primer | GCCTGTCGATTATACAGATGATGAAA | (SEQ ID NO:22) | 1660 | |
| Reverse Primer | CGCTGCCCAGTGTTCATTACT | (SEQ ID NO:23) | 1767 | |
| Probe | AGAAGAATGAATCATCCTCAGCTAGCCACTGCT | (SEQ ID NO:24) | 1713 | |

Primers and probes are shown in the 5′ to 3′ direction.

The cDNA amplification reactions were performed in 25 μL volumes containing dATP, dCTP, and dGTP (0.2 mM each), dUTP (0.4 mM), Amplitaq Gold (0.625 units), Amperase UNG (0.25 units), forward primer (300 nM), reverse primer (300 nM), and probe (TIEG, Smad 2, Smad 7 and BARD-1, 160 nM; β-actin, 200 nM). In addition, trace amounts of glycerol, Tween 20, and glycine were added to stabilize each reaction. Further, each reaction contained 5 μL of diluted (1:100) cDNA template from the RT reaction or 5 μL of water (as a no template control). Each reaction was performed in duplicate. Primers and probes for β-actin (see Table 3) were used in a similar manner to quantitate the presence of β-actin mRNA as an endogenous RNA control in the samples. Retropseudogenes of β-actin that lead to coamplification of contaminating genomic DNA were not detected in control reactions, as no signal was observed with PCR amplification of RT reaction products that contained no reverse transcriptase. The universal thermal cycling program for cDNA amplification by real-time PCR consisted of an initial 2-minute incubation at 50° C. to activate the UNG enzyme, a 10-minute incubation at 95° C. to activate the Amplitaq Gold, and 40 cycles at 95° C. for 0.15 minutes and 60° C. for 1 minute.

TIEG, Smad 2, Smad 7, BARD-1, and β-actin standard curves were generated using Ct values determined from a series of cDNA amplification reactions identical to those described above, except that each assay contained a standard dilution of control plasmid containing full-length TIEG, Smad 2, Smad 7, BARD-1, or β-actin. Standard dilutions ranging from 1 to 1×10⁴ copies/μL of TIEG, Smad 7, or BARD-1 plasmid, or 100 to 1×10⁶ copies/μL (0.032 pg/mL to 312 pg/mL) of β-actin plasmid were added to each reaction. The β-actin standard curve was generated to normalize each TIEG cDNA amplification product to a constant amount of β-actin mRNA.

The sequence of each cDNA amplification product was confirmed by DNA sequencing. Briefly, 25 μL of the cDNA amplification reaction product was mixed with 2.5 μL of sample loading buffer. Each sample was separated on a 2% (w/v) Seakem GTG agarose gel in 1×TBE at 5 v/cm. The separated bands were visualized by staining the gel with Sybr Green I for 45 minutes. The gel was then photographed with a digital camera (Eastman Kodak, Rochester, N.Y.). After photographing the gel, the bands of interest (see Table 3 for product band sizes) for each gene were excised, and the corresponding DNA was extracted (QIAquick® gel extraction kit; Qiagen) and submitted for automated DNA sequence analysis. The Wisconsin Sequence Analysis Package (version 10 for UNIX; Genetics Computer Group, Madison, Wis.) was used to analyze the sequence data and confirm the sequences for each gene.

TIEG

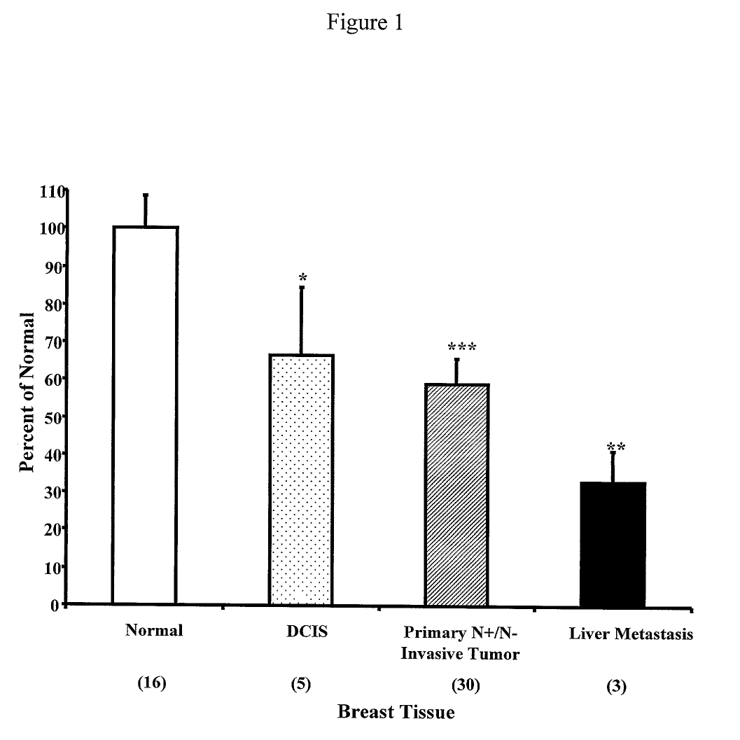

Student's t-test evaluation of primary tumor TIEG gene expression (as assessed using the TIEG80 primers and probe in Table 3) and known pathological characteristics revealed no significant correlations between TIEG mRNA copy number and histology, tumor size, and hormone receptor status of the primary tumors (Table 4). The average TIEG mRNA level, however, was 58% higher in Her2-negative cancers compared to Her2-positive cancers (Table 4). TIEG gene expression levels were significantly different between N− and N+ patients. The average TIEG gene expression level was 58% higher in N− versus N+ tumors (Table 4), although both types still contained significantly less TIEG mRNA than normal breast tissue (FIG. 1).

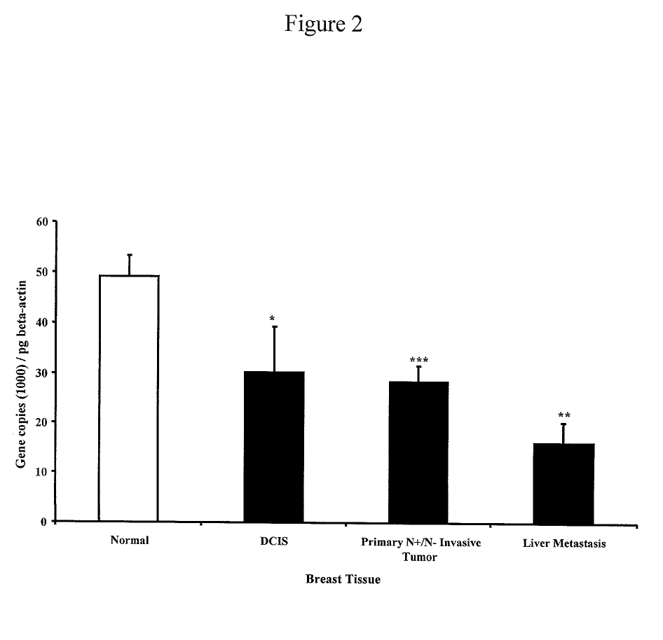

The average TIEG gene expression level (as assessed using the TIEG80 primers and probe in Table 3) was reduced by 37%, 42%, and over 67% in non-invasive (DCIS), invasive, and metastatic breast tumor tissue (liver metastasis), respectively, compared to normal breast tissue (FIGS. 1 and 2). These findings demonstrate that an inverse correlation exists between TIEG gene expression levels and breast cancer disease progression.

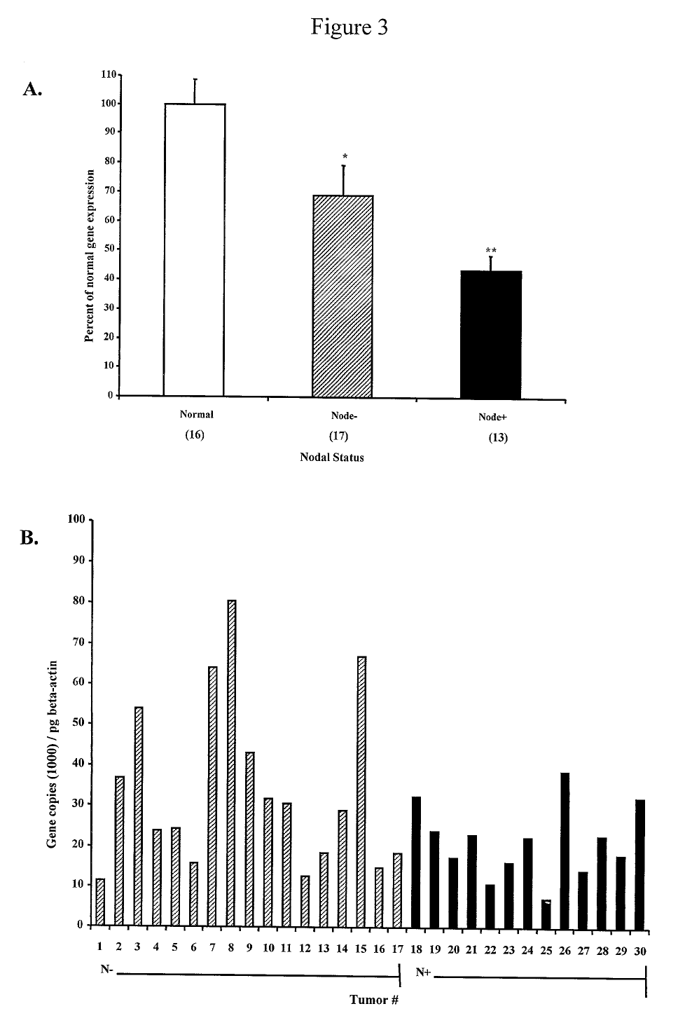

Real-time PCR analysis revealed that TIEG gene expression levels (as assessed using the TIEG80 primers and probe in Table 3) in N− and N+ primary tumors can be used to differentiate nodal status in primary breast tumor patients. As shown in FIG. 3A, both N− and N+ tumors had significantly lower average TIEG gene expression levels compared to normal breast tissue. Furthermore, the average N− TIEG gene expression level was 58% higher than the average N+ TIEG gene expression level (average N−versus N+: p=0.055 (t-test), p=0.11 (Wilcoxon)). The data shown in FIG. 3B demonstrate that individual N− tumors have, on average, higher TIEG mNRA levels compared to individual N+ tumors.

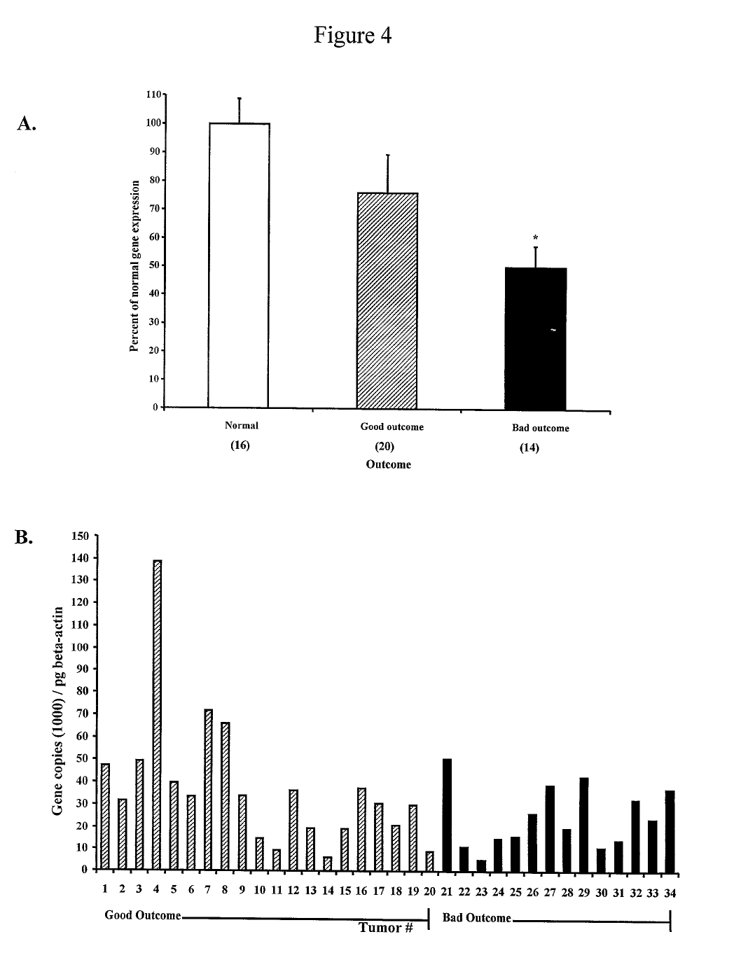

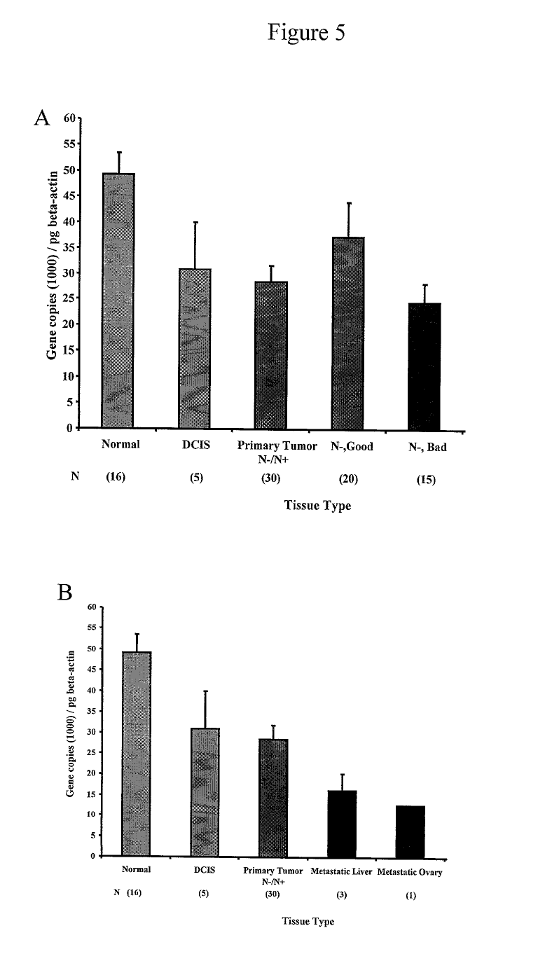

In addition, real-time PCR analysis revealed that the average TIEG gene expression level (as assessed using the TIEG80 primers and probe in Table 3) in N−, bad outcome samples was 50% lower than that in normal breast tissue samples, whereas the average TIEG gene expression level in N−, good outcome samples was only 24% lower (FIG. 4A). Furthermore, the average N−, good outcome TIEG gene expression level was 52% higher than the average N−, bad outcome TIEG gene expression level (average N−, good versus N−, bad: p=0.21 (t-test), p=0.23 (Wilcoxon)). The data shown in FIG. 4B demonstrate that individual N−, good outcome tumors have, on average, higher TIEG mRNA levels compared to individual N−, bad outcome tumors. Although the TIEG gene expression level averages between the two groups were not significantly different at an alpha of 0.05, a trend was present that suggests N−, good outcome tumors could have higher TIEG mRNA levels compared to N−, bad outcome tumors (see FIG. 4B). FIGS. 5A and 5B show further data indicating that TIEG gene expression levels are lower in cancerous and metastatic tissues compared to the levels in normal tissues.

TABLE 4
Clinicopathological characteristics of breast cancer patients used
for TIEG, Smad 7, Smad 2, and BARD-1 analysis

| | | TIEG80 | | | Smad 7 | | | Smad 2 | | | BARD-1 | | |
|---|---|---|---|---|---|---|---|---|---|---|---|---|---|
| | n | mean | SEM | P | mean | SEM | P | mean | SEM | P | mean | SEM | P |
| Histology | 30 | | | 0.22 | | | 0.099 | | | 0.6 | | | 0.97 |
| Ductal | 23 | 25.8 | 3.1 | | 9.63 | 1.28 | | 18 | 2.82 | | 2.81 | 0.482 | |
| Lobular | 6 | 35.9 | 11 | | 16.3 | 5.87 | | 15 | 3.22 | | 2.84 | 0.368 | |
| S | 1 | 43 | | | 5.183 | | | 15.5 | | | 1.75 | | |
| Tumor | 30 | | | 0.75* | | | 0.98* | | | 0.25* | | | 0.66* |
| Size | | | | | | | | | | | | | |
| T1b, c | 12 | 34.2 | 5.8 | | 13.6 | 3.77 | | 24.1 | 4.48 | | 3.44 | 0.878 | |
| T2 | 14 | 22.1 | 2.25 | | 8.2 | 0.764 | | 12.3 | 1.06 | | 2.21 | 0.235 | |
| T3 | 4 | 36.1 | 15.3 | | 12.6 | 4.28 | | 14.4 | 3.31 | | 2.73 | 0.609 | |
| Lymph | 30 | | | 0.055 | | | 0.17 | | | 0.048 | | 0.048 | 0.13 |
| Node | | | | | | | | | | | | | |
| Positive | 13 | 21.4 | 2.51 | | 8.97 | 1.23 | | 12.5 | 1.02 | | 2.32 | 0.18 | |
| Negative | 17 | 33.8 | 5.09 | | 12.6 | 2.82 | | 20.9 | 3.45 | | 4.21 | 1.06 | |
| Her2 | 30 | | | 0.052 | | | 0.12 | | | 0.23 | | | 0.068 |
| Positive | 12 | 21.1 | 2.5 | | 7.4 | 0.956 | | 14.2 | 2.16 | | 1.94 | 0.226 | |
| Negative | 18 | 33.3 | 4.88 | | 13.3 | 2.52 | | 19.4 | 3.18 | | 3.33 | 0.577 | |
| ER | 30 | | | 0.8 | | | 0.46 | | | 0.47 | | | 0.64 |
| Positive | 24 | 28 | 4.01 | | 11.7 | 2.03 | | 18.1 | 2.61 | | 2.86 | 0.461 | |
| Negative | 6 | 30.1 | 3.13 | | 8.11 | 0.931 | | 14.2 | 1.66 | | 2.42 | 0.386 | |
| PR | 30 | | | 0.98 | | | 0.51 | | | 0.31 | | | 0.53 |
| Positive | 25 | 28.4 | 3.76 | | 11.5 | 1.94 | | 18.3 | 2.47 | | 2.88 | 0.44 | |
| Negative | 5 | 28.6 | 5.89 | | 8.1 | 2.77 | | 12.3 | 2.37 | | 2.24 | 0.495 | |

P values in parentheses are Student's t values with respect to normal.
*p value for T3 versus T1. Values are expressed as copies × 1000/pg β-actin mRNA.

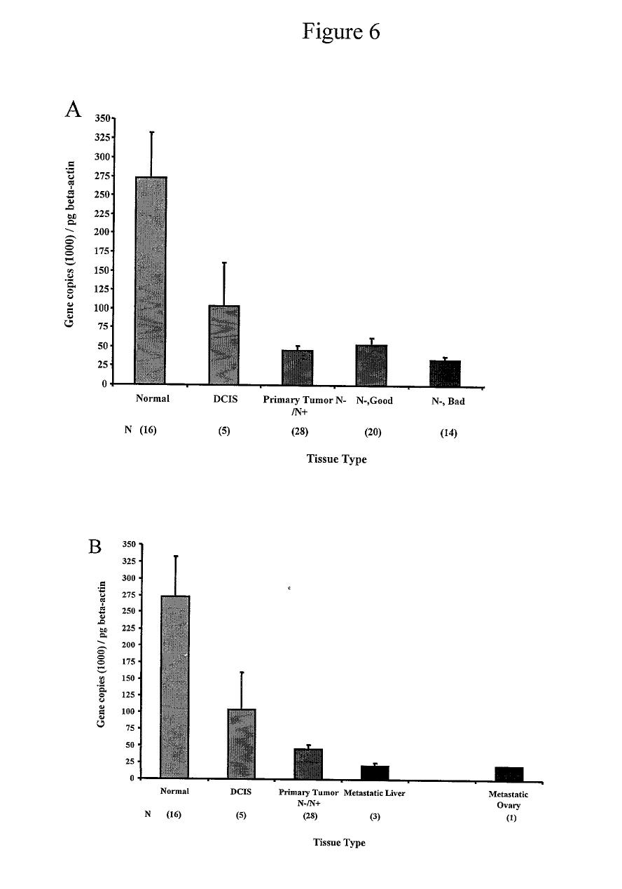

Because TIEG is mainly localized to epithelial cells, TIEG80 gene expression was adjusted for epithelial content. Epithelial content was determined by estimating the percentage of epithelial cells found in a tissue section stained with hematoxylin and eosin. TIEG80 gene expression (gene copies×1000/pg β-actin mRNA) was then divided by the epithelial content percentage. The results are listed in Table 5 and shown in FIGS. 6A and 6B. Additional data describing the correlation of TIEG gene expression in epithelial corrected samples with nodal status and patient outcome are presented in Table 8.

Examination of TIEG gene expression levels by amplifying the gene regions that code for mid, N-terminal, and C-terminal zinc-finger regions of the TIEG protein revealed that TIEG gene expression levels were also higher in N− versus N+ primary breast tumors (Table 6). Furthermore, TIEG mNRA levels measured under these conditions were higher in N−, good outcome samples versus N−, bad outcome samples.

In summary, these data demonstrate that breast cancer samples contain significantly less TIEG mRNA than normal breast tissue samples. In addition, these data demonstrate that an inverse correlation exists between TIEG gene expression levels and breast cancer disease progression. These data also demonstrate that N−, bad outcome tumors have a reduced levels of TIEG mRNA compared to N−, good outcome tumors. Further, these data indicate that TIEG mRNA levels can be correlated with cancer progression and patient outcome.

TABLE 5
Clinicopathological characteristics of breast cancer patients
used for TIEG epithelial cell corrected studies.

| | | TIEG80 | | |
|---|---|---|---|---|
| | n | mean | SEM | P |
| Histology | 28 | | | 0.20 |
| Ductal | 21 | 39.59 | 4.93 | |
| Lobular | 6 | 57.1 | 18.97 | |
| S | 1 | 107.5 | | |
| Tumor Size | 28 | | | 0.59 |
| T1b, c | 11 | 45.44 | 9.17 | |
| T2 | 13 | 42.5 | 7.08 | |
| T3 | 4 | 57.3 | 26.8 | |
| Lymph Node | 28 | | | 0.03 |
| Positive | 12 | 31.5 | 4.90 | |
| Negative | 16 | 56.5 | 8.27 | |
| Her2 | 28 | | | 0.10 |
| Positive | 11 | 33.8 | 5.23 | |
| Negative | 17 | 53.5 | 8.74 | |
| ER | 28 | | | 0.01 |
| Positive | 22 | 39.01 | 5.90 | |
| Negative | 6 | 70.54 | 12.7 | |
| PR | 28 | | | 0.15 |
| Positive | 23 | 41.6 | 5.66 | |
| Negative | 5 | 64.91 | 18.98 | |

TABLE 6
TIEG gene expression with other gene regions amplified.

| | TIEG515 | TIEG529 | TIEG1217 |
|---|---|---|---|
| % higher N− vs. N+ | 348 ± 123 | 119 ± 56 | 123 ± 53 |
| Student's t-test p-value | 0.05 | 0.08 | 0.057 |
| Wilcoxon Rank-sum test p-value | 0.012 | 0.035 | 0.028 |
| % higher N−, good vs. N−, bad | 150 ± 75 | 33 ± 26 | 12 ± 18 |
| Student's t-test p-value | 0.099 | 0.361 | 0.646 |
| Wilcoxon Rank-sum test p-value | 0.139 | 0.283 | 0.564 |

Values represent the average percent difference between N-versus N+ and between N−, good outcome and N−, bad outcome primary breast cancer patients ± standard error of the mean.

BARD-1

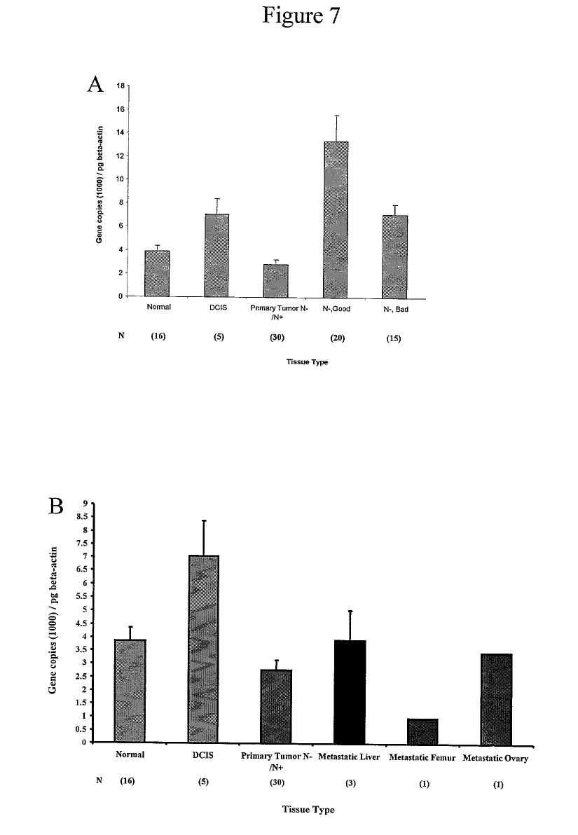

Real-time PCR analysis of BARD-1 mRNA levels in breast cancer samples revealed that BARD-1 analysis can be useful in determining cancer aggressiveness. BARD-1 analysis data are listed in Table 4. BARD-1 gene expression in various tissues is shown in FIGS. 7A and 7B. Additional data describing the correlation of BARD-1 gene expression with nodal status and patient outcome are presented in Table 8. These data demonstrate that the level of BARD-1 mNRA in a sample can be correlated with cancer aggressiveness. Specifically, a reduced level of BARD-1 mRNA in a breast cancer sample indicates that breast cancer is aggressive.

Smad 7

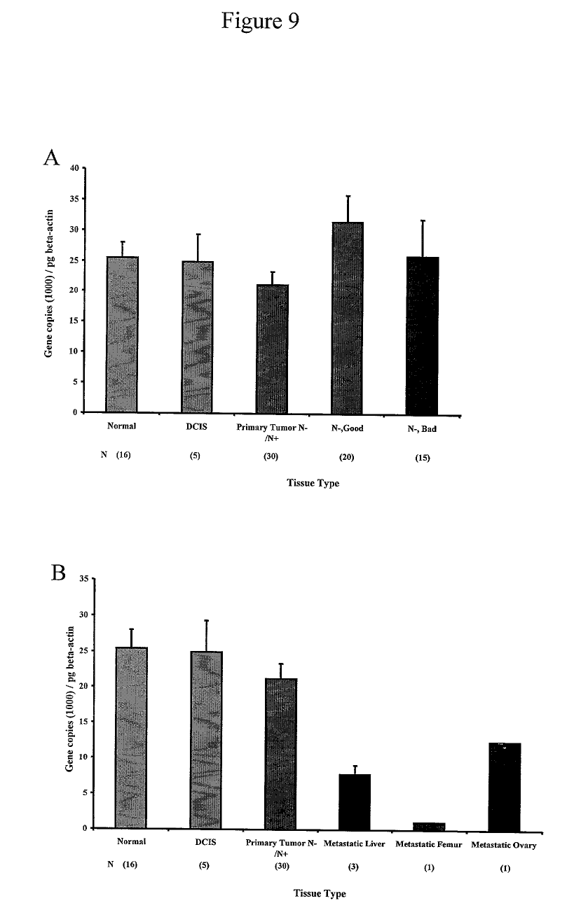

Smad 7 gene expression data revealed a difference in mRNA levels between N+ and N− primary breast tumors. N+ samples exhibited a lower level of mRNA, while N− samples exhibited a higher level of mNRA (Table 4; FIGS. 9A and 9B). In addition, the average level of gene expression in both N+ and N− patient groups is higher than the level of gene expression in normal patient group. No difference in gene expression between N−, good and N−, bad primary breast tumors was observed. Further, no correlation between gene expression and N−, good or N+, bad outcomes was observed. Additional data describing the correlation of Smad 7 gene expression with nodal status and patient outcome are presented in Table 7. These data demonstrate that Smad 7 mRNA levels can be used to differentiate N+ and N− primary breast tumors. These data also demonstrate that an elevated level of Smad 7 mRNA in a test sample indicates the presence of cancer.

Smad 2

Smad 2 gene expression data revealed a difference in mNRA levels between N+ and N− primary breast tumors. N+ samples exhibited a lower level of mNRA, while N− samples exhibited a higher level of mRNA (Table 4; FIGS. 10A and 10B). In addition, the average level of gene expression in both N+ and N− patient groups is lower than the level of gene expression in normal patient group. No difference in gene expression between N−, good and N−, bad primary breast tumors was observed. Further, no correlation between gene expression and N−, good or N+, bad outcomes was observed. Additional data describing the correlation of Smad 2 gene expression with nodal status and patient outcome are presented in Table 7. These data demonstrate that Smad 2 mRNA levels can be used to differentiate N+ and N− primary breast tumors. These data also demonstrate that a reduced level of Smad 2 mRNA in a test sample indicates the presence of cancer.

TIEG Marker Combinations

Experiments were performed to test the ability of multiple markers to correlate with prognosis and patient outcome. TIEG markers were tested in various patient samples as described above. The data presented in Table 7 indicate that combinations of markers (e.g., TIEG/BARD-1) provide good correlative information with nodal status and patient outcome

TABLE 7
Sensitivity/specificity results for TIEG markers

| | | Comparison Group (sensitivity/specificity) | | |
|---|---|---|---|---|
| | | Normal vs. Tumor | | N−/good vs. N−/ |
| Gene(s) | | (N−/N+) | N− vs. N+ | bad |
| TIEG80epi* | high = better | 89/87 | 83/69 | 64/60 |
| Smad7 | low = better | 60/61 | 62/47 | 40/40 |
| Smad2 | high = better | 73/67 | 77/59 | 67/80 |
| BARD-1 | high = better | 70/60 | 62/65 | 80/80 |
| TIEG80epi*/BARD-1 | at least 1 | 71/100 | 83/75 | 79/90 |
| | high = better | | | |
| TIEG80/Smad 7 | | | 77/100 | 79/80 |
| TIEG80/Smad2 | | | 100/77 | 79/80 |
| TIEG80/BARD-1 | | | 77/100 | 93/80 |

*TIEG80epi values are corrected for epithelial cell content

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the aggressiveness of a cancer in a mammal, said method comprising determining, in a test sample from said mammal, the presence or absence of a TIEG marker and correlating said presence or absence with said aggressiveness.

2. The method of claim 1, wherein said cancer is breast cancer.

3. The method of claim 1, wherein said mammal is a human.

4. The method of claim 1, wherein said test sample is a tumor biopsy.

5. The method of claim 1, wherein said presence of said marker indicates said cancer is aggressive.

6. The method of claim 1, wherein said presence of said marker indicates said cancer is metastatic.

7. The method of claim 1, wherein said absence of said marker indicates said cancer is not aggressive.

8. The method of claim 1, wherein said marker is a reduced level of TIEG RNA in said test sample compared to a control level of TIEG RNA.

9. The method of claim 1, wherein said marker is a reduced level of Smad 2 RNA in said test sample compared to a control level of Smad 2 RNA.

10. The method of claim 1, wherein said marker is an elevated level of Smad 7 RNA in said test sample compared to a control level of Smad 7 RNA.

11. The method of claim 1, wherein said marker is a reduced level of BARD-1 RNA in said test sample compared to a control level of BARD-1 RNA.

12. The method of claim 1, wherein said method comprises determining, in said test sample from said mammal, the presence or absence of two TIEG markers.

13. The method of claim 12, wherein said two markers are a reduced level of TIEG RNA in said test sample compared to a control level of TIEG RNA and a reduced level of Smad 2 RNA in said test sample compared to a control level of Smad 2 RNA.

14. The method of claim 12, wherein said two markers are a reduced level of TIEG RNA in said test sample compared to a control level of TIEG RNA and an elevated level of Smad 7 RNA in said test sample compared to a control level of Smad 7 RNA.

15. The method of claim 12, wherein said two markers are a reduced level of TIEG RNA in said test sample compared to a control level of TIEG RNA and a reduced level of BARD-1 RNA in said test sample compared to a control level of BARD-1 RNA.

16. The method of claim 12, wherein said two markers are a reduced level of BARD-1 RNA in said test sample compared to a control level of BARD-1 RNA and an elevated level of Smad 7 RNA in said test sample compared to a control level of Smad 7 RNA.

17. The method of claim 12, wherein said two markers are a reduced level of Smad 2 RNA in said test sample compared to a control level of Smad 2 RNA and an elevated level of Smad 7 RNA in said test sample compared to a control level of Smad 7 RNA.

18. The method of claim 1, wherein said method comprises determining, in said test sample from said mammal, the presence or absence of three TIEG markers.

19. The method of claim 18, wherein said three markers comprise a reduced level of TIEG RNA in said test sample compared to a control level of TIEG RNA, a reduced level of BARD-1 RNA in said test sample compared to a control level of BARD-1 RNA, and a reduced level of SMAD-2 RNA in said sample compared to a control level of SMAD-2 RNA

20. The method of claim 18, wherein said three markers comprise a reduced level of TIEG RNA in said test sample compared to a control level of TIEG RNA, a reduced level of BARD-1 RNA in said test sample compared to a control level of BARD-1 RNA, and an elevated level of SMAD-7 RNA in said sample compared to a control level of SMAD-7 RNA.

21. The method of claim 18, wherein said three markers comprise a reduced level of TIEG RNA in said test sample compared to a control level of TIEG RNA, a reduced level of SMAD-2 RNA in said test sample compared to a control level of SMAD-2 RNA, and an elevated level of SMAD-7 RNA in said sample compared to a control level of SMAD-7 RNA.

22. The method of claim 1, wherein said marker is measured in comparison to a baseline established for said cancer.

23. The method of claim 22, wherein said marker is a reduced level of TIEG RNA, wherein said baseline is from about 89 to about 100 mRNA molecules ×1000 per picogram of beta-actin mRNA, and wherein said cancer is breast cancer.

24. An article of manufacture comprising an oligonucleotide primer pair that specifically amplifies all or a portion of a target region of a TIEG nucleic acid, wherein said target region is defined by nucleotides 1-500 of the 5′ portion of said TIEG nucleic acid.

25. The article of manufacture of claim 24, wherein said target region is defined by nucleotides 80-188 of said TIEG nucleic acid.

26. The article of manufacture of claim 25, wherein said primer pair specifically amplifies all of said target region defined by nucleotides 80-188 of said TIEG nucleic acid.

27. An article of manufacture comprising a first and a second oligonucleotide primer pair, wherein said first primer pair amplifies a first target nucleic acid and said second primer pair amplifies a second target nucleic acid in an amplification reaction, and wherein said first and second target nucleic acids are TIEG marker-related nucleic acids.

28. The article of manufacture of claim 27, wherein said first target nucleic acid is a TIEG nucleic acid and said second target nucleic acid is a Smad 2 nucleic acid.

29. The article of manufacture of claim 27, wherein said first target nucleic acid is a TIEG nucleic acid and said second target nucleic acid is a Smad 7 nucleic acid.

30. The article of manufacture of claim 27, wherein said first target nucleic acid is a TIEG nucleic acid and said second target nucleic acid is a BARD-1 nucleic acid.

31. The article of manufacture of claim 27, wherein said first target nucleic acid is a TIEG nucleic acid and said second target nucleic acid is a nucleic acid encoding a biomolecule regulated by TIEG.

32. The article of manufacture of claim 27, further comprising a third oligonucleotide primer pair, wherein said third primer pair amplifies a third target nucleic acid in an amplification reaction, and wherein said third target nucleic acid is a TIEG marker-related nucleic acid.

33. The article of manufacture of claim 32, wherein said first target nucleic acid is a TIEG nucleic acid, said second target nucleic acid is a BARD-1 nucleic acid, and said third target nucleic acid is a Smad 2 nucleic acid.

34. The article of manufacture of claim 32, wherein said first target nucleic acid is a TIEG nucleic acid, said second target nucleic acid is a BARD-1 nucleic acid, and said third target nucleic acid is a Smad 7 nucleic acid.

35. The article of manufacture of claim 32, wherein said first target nucleic acid is a TIEG nucleic acid, said second target nucleic acid is a Smad 2 nucleic acid, and said third target nucleic acid is a Smad 7 nucleic acid.

36. The article of manufacture of claim 27, further comprising an oligonucleotide probe specifically hybridizable to a TIEG marker-related nucleic acid.

37. The article of manufacture of claim 27, further comprising a label or package insert indicating that a level of a TIEG marker-related nucleic acid in a test sample from a mammal can be correlated with aggressiveness of a cancer in said mammal.

38. The article of manufacture of claim 37, wherein said label further comprises a baseline level or levels established for said cancer.

39. The article of manufacture of claim 27 further comprising a label or package insert indicating that a test sample from a mammal can be adjusted for epithelial cell content prior to determining the level of a TIEG marker-related nucleic acid in said test sample.

40. An antibody having specific binding affinity for a TIEG polypeptide.

41. The antibody of claim 40, wherein the amino acid sequence of said TIEG polypeptide comprises a sequence selected from the group consisting SEQ ID NO:25, 26, 27, 28, and 29.

42. A method for determining the aggressiveness of a cancer in a mammal, said method comprising contacting the antibody of claim 40 with a test sample from said mammal, detecting the presence or absence of complexes between said antibody and a TIEG polypeptide, and correlating said presence or absence with said aggressiveness.

43. The method of claim 42, further comprising determining the presence or absence of a TIEG marker other than TIEG polypeptide in said sample.

44. A method of assisting a person in determining the aggressiveness of a cancer in a mammal, wherein said method comprises:
a) determining the presence or absence of a TIEG marker in a sample from said mammal; and,
b) communicating information about said presence or absence of said marker in said sample to said person.

45. A method for determining the prognosis of a mammal having a cancer, said method comprising determining, in a test sample from said mammal, the presence or absence of a TIEG marker and correlating said presence or absence with said prognosis.

46. The method of claim 45, wherein said presence of said TIEG marker indicates that said prognosis of said mammal is a bad outcome.

47. The method of claim 45, wherein said absence of said TIEG marker indicates that said prognosis of said mammal is a good outcome.

48. A method for diagnosing cancer in a mammal, said method comprising determining, in a test sample from said mammal, the presence or absence of a TIEG marker and correlating said presence or absence with said diagnosis.

49. The method of claim 48, wherein said presence of said marker indicates a diagnosis of cancer.

50. The method of claim 48, wherein said absence of said marker indicates a diagnosis of no cancer.

51. The method of claim 48, wherein said marker is a reduced level of TIEG RNA in said test sample compared to a control level of TIEG RNA.

52. The method of claim 1, wherein said test sample from said mammal comprises at least 60% epithelial cells.

53. The method of claim 1, wherein said marker is adjusted to reflect epithelial cell content of said test sample.

54. The article of manufacture of claim 24, further comprising a label or package insert indicating that a level of a TIEG nucleic acid in a test sample from a mammal can be correlated with aggressiveness of a cancer in said mammal.

55. A method for determining the aggressiveness of a cancer in a mammal, said method comprising determining, in a test sample from said mammal, the level of a TIEG biomolecule and the level of a biomolecule regulated by TIEG, and correlating said levels with said aggressiveness.
